(19) United States
(12) Patent Application Publication
(10) Pub. No.: US 2026/0345485 A1
(43) Pub. Date: Nov. 5, 2026

(54) INTRATHECAL NANOPARTICLE DELIVERY FOR TREATMENT OF LEPTOMENINGEAL TUMORS WITH CORE-SHELL PARTICLES MADE OF HYPERBRANCHED POLYGLYCEROL AND POLYLACTIC ACID

(71) Applicant: YALE UNIVERSITY, New Haven, CT (US)

(72) Inventors: Minsoo KHANG, New Haven, CT (US); Ranjit S. BINDRA, New Haven, CT (US); W. Mark SALTZMAN, New Haven, CT (US)

(21) Appl. No.: 19/122,263

(22) PCT Filed: Nov. 9, 2023
(86) PCT No.: PCT/US2023/079181

§ 371 Date: Apr. 18, 2025

Related U.S. Application Data
(60) Provisional application No. 63/383,211, filed on Nov. 10, 2022.

(51) Int. Cl. A61K 9/00; A61K 9/51; A61K 31/495; A61K 45/06; A61P 35/00

(52) U.S. Cl. CPC A61K 9/0085; A61K 9/0019; A61K 9/5146; A61K 31/495; A61K 45/06; A61P 35/00

(57) ABSTRACT

Bioadhesive biodegradable polymeric nanoparticles can be administered intrathecally into the spinal column, for example, through the cisterna magna, for dissemination for the treatment of tumors such as leptomeningeal metastasis. The nanoparticles rapidly spread to all central spinal fluid (CSF) compartments, including the brain parenchyma and spinal column. The bioadhesive nanoparticles penetrate and are retained for long periods, during which they can continue to release agents. These nanoparticles can be loaded with different therapeutic, prophylactic or diagnostic agent, most preferably DNA-repair inhibitor drugs to enhance killing of leptomeningeal tumors like leptomeningeal metastasis and seeding tumors such as medulloblastoma. In a preferred embodiment, the patients are treated with a combination of a PARP inhibitor and temozolomide.

### CROSS REFERENCE TO RELATED APPLICATION

This application claims the benefit of and priority to U.S. Provisional Application No. 63/383,211 filed Nov. 10, 2022, which is hereby incorporation by reference in its entirety.

### STATEMENT REGARDING FEDERALLY SPONSORED RESEARCH

This invention was made with government support under CA149128 awarded by the National Institutes of Health. The government has certain rights in the invention.

### FIELD OF THE INVENTION

This technology is generally related to formulations for treatment of brain cancers by direct intrathecal administration to the cerebral spinal fluid.

### BACKGROUND OF THE INVENTION

Medulloblastoma (MB) is the most common malignant brain tumor in children. Medulloblastoma is a malignant brain tumor that starts in the lower back part of the brain, called the cerebellum. The cerebellum is involved in muscle coordination, balance and movement. Medulloblastoma tends to spread through cerebrospinal fluid (CSF), the fluid that surrounds and protects the brain and spinal cord, to other areas around the brain and spinal cord. This tumor rarely spreads to other areas of the body. Medulloblastoma can occur at any age, but most often occurs in young children. Though medulloblastoma is rare, it's the most common cancerous brain tumor in children.

Treatment for medulloblastoma usually includes surgery followed by radiation or chemotherapy, or both. Age and general health, tumor subtype and location, tumor grade and extent, and other factors play a role in treatment decisions. Options include surgery to remove the medulloblastoma. However, sometimes it is not possible to remove the tumor entirely because medulloblastoma forms near critical structures deep within the brain. All patients with medulloblastoma should receive additional treatments after surgery to target any remaining cells.

Craiospinal Radiation therapy (CSI) is another option. A pediatric or adult radiation oncologist administers radiation therapy to the brain and spinal cord using high-energy beams, such as X-rays or protons, to kill cancer cells. Standard radiation therapy can be used, but proton beam therapy delivers higher targeted doses of radiation to brain tumors, minimizing radiation exposure to nearby healthy tissue. This has the highest survival benefit, but causes 30-40% decreases in intelligence quotients at the average dose of 1000 mGy, with more at 10 Gy or higher. The average for CSI is 23-36 Gy. Moreover, 40% of the children have tumor recurrence, with a five year survival rate of only 60%. CSI causes DNA damage in all cells, not just the tumor cells.

Chemotherapy is an alternative. Typically, children and adults with medulloblastoma receive these drugs as an injection into the vein (intravenous chemotherapy). Chemotherapy may be recommended after surgery or radiation therapy, or in certain cases, at the same time as radiation therapy. In some cases, high dose chemotherapy followed by stem cell rescue (a stem cell transplant using the patient's own stem cells) may be used.

Leptomeningeal spread commonly occurs in multiple subsets of pediatric central nervous system tumors, and as metastasis from adult solid tumors. Current treatment regimens have undesirable side-effects, and do not improve leptomeninges prognosis significantly. One can treat occult cells in the cerebral spinal fluid (“CSF:) and in leptomeningeal cancers, but it is associated with severe cognitive late effects from brain parenchymal exposure to radiotherapy. However, the most common sites of relapse are the CSF and the leptomeninges, so delivering treatment directly to those sites can reduce brain and tissue toxicity. Intrathecal delivery has the advantage of bypassing the blood-brain barrier and limiting systemic normal tissue toxicity, but intrathecally delivered drugs clear too quickly from the CSF to be effective.

### SUMMARY OF THE INVENTION

Polymeric nanoparticles (NPs) are administered intrathecally (through the cisterna magna) for central nervous system dissemination of radiosensitizers or other chemotherapeutic agents, and/or diagnostic/imaging agents for the treatment of cancers such as medulloblastomas and leptomenigeal tumors. When fluorescently labeled NPs are injected into the cisterna magna, there is rapid spread to all CSF compartments, including the brain parenchyma and spinal column. Bioadhesive nanoparticles also penetrate, and are retained for long periods, during which they can continue to release agents. These nanoparticles can be loaded with different DNA-repair inhibitor drugs to enhance killing of leptomeningeal tumors (like leptomeningeal metastasis) and seeding tumors (such as medulloblastoma)

The NPs are made from a hyperbranched polyglycerol-polyhydroxyacid polymer that has bio-adhesive properties, which allows the NPs to selectively bind to tumor cells relative to normal microglia and neuron cells. Comparison of the zeta potential (mV) of the bioadhesive HPG NPs (“BNPs”) having amine groups on the surface demonstrated the BNPs have a significantly more negative potential (−28.4 mV for non-adhesive PLA-HPG NPs (“NNPs”) versus-47.2 mV for PLA-HPG BNPs. Small molecular weight compounds, such as the imaging agent DFO can be conjugated to the surfaces of the BNPs, with altering stability or bioadhesiveness. Loading capacities for low molecular agent was in the range of 3%, up to 5%, depending on the drug to polymer or solvent ratio.

The results described herein uses a PLA-HPG NP platform that remains in the subarachnoid region for long periods of times, in contrast to the fate of freely administered small molecules. Long-term retention of the NPs in the CSF space of tumor-free mice, as well as preferential accumulation and retention in CSF-adjacent tumors, was demonstrated using PET/CT and fluorescent whole-body imaging. Increasing accumulation of nanoparticles in the tumor early-on in circulation lessens the probability of mononuclear phagocytic system and renal system clearance.

The vast majority of polymeric NPs exhibit substantial absorption in the spleen and clearance organs such as the liver and kidney, potentially limiting their therapeutic use. Results demonstrated that PLA-HPG NPs retain their ideal qualities in the CSF space as well, with the hyperbranched structure forming a steric barrier around the NPs that extends circulation time and allows for greater tumor site accumulation. Significant accumulation of NPs in the meninges of tumor-bearing mice, as well as at the site of tumor in the cerebellum, was observed. With controlled release of drug, long-term retention of NPs can lead to prolonged drug exposure at site of tumor, and a means of improving the overall half-life of drug following intra-CSF administration. In addition, because activity in the CNS is at least 75% of total activity at all time points measured, there is a greatly reduced risk of widespread systemic toxicity.

PARP inhibitors are limited by BBB penetration and widespread toxicity. PARPi were initially developed to sensitize tumor cells to conventional DNA damaging agents, and increasing evidence shows that PARPi is effective at sensitizing cells to radiotherapy, to temozolomide, and to topoisomerase poisons and inhibitors. Talazoparib (BMN-673) is a potent PARPI trapper, but is constrained by its inability to bypass the BBB in meaningful quantities. Studies with BMN-673 are the first known preclinical study of delivering PARPi intrathecally, and unacceptable levels of toxicity in the free drug both alone and in combination with TMZ were found. Nanoencapsulation significantly improves the therapeutic index of BMN-673. Significantly higher doses (10×) with less systemic toxicity compared to free drug, as measured by blood cell counts, weight loss, and organ toxicity, were possible. Single-agent efficacy in an orthotopic model of MB was measured by giving equitoxic doses of either BMN-673 or BMN-NPs, and it was observed that only the encapsulated BMN NPs led to consistent tumor regression and an overall decrease in leptomeningeal spread. In addition, BMN-NPs were administered in conjunction with low-dose TMZ, and it was observed that this combination led to a durable response and was well-tolerated by the mice. This integrated treatment approach provides new opportunities for PARPi combination therapies without compromising tolerability. In addition, this approach could lead to promising avenues of treatment for other diseases associated with extensive leptomeningeal spread, such as leptomeningeal metastases from primary malignancies such as lung, breast, and melanoma cancer.

### BRIEF DESCRIPTION OF THE DRAWINGS

FIG. 1A is a cross-sectional schematic of a brain showing the skull, blood vessels, cancer cells, cerebral spinal fluid (CSF), and brain. FIG. 1B is a cross-sectional schematic of the periarterial space, which the NPs disperse through. These show the location in the cisterna magna of the subarachnoid space where the dye-loaded nanoparticles were injected and distributed to. The CSF drains through both arachnoid granulations and lymphatics.

FIG. 2A is a cross-sectional schematic of a polylactic acid (PLA)-hyperbranched polyglycerol (HPG) NP, having bioadhesive aldehyde groups on the surface. FIG. 2B is schematic of the synthesis of the NP of FIG. 2A.

FIGS. 3A-3C are schematics of the chemical structure of PLA-HPG NPs with DFO conjugation, and DFO-⁸⁹Zr binding (FIG. 3A); Reaction process showing conjugation of DFO onto NPs (FIG. 3B), and stability of NPs (consistent PDI and Z-average) measured by dynamic light scattering (n=3) (FIG. 3C). DFO conjugation does not affect bioadhesive properties of NPs, as measured by a poly-lysine assay.

FIGS. 4A-4C are graphs of PET/CT of ⁸⁹Zr-DFO-NPs and ⁸⁹Zr-DFO delivered ICM in tumor-free mice. n=2 healthy mice were injected with either of ⁸⁹Zr-DFO-NPs and 89-Zr-DFO and imaged continuously for 2 hours. Pharmacokinetic curve in the CNS of ⁸⁹Zr-DFO-NPs and 89-Zr-DFO, *P=0.0243 (FIG. 4A). Pharmacokinetic curve in the brain and spinal cord separately of ⁸⁹Zr-DFO-NPs and ⁸⁹Zr-DFO, ****P<0.0001 (brain), ns (spinal cord) (FIG. 4B). Based on whole-body sagittal plane PET images of continuous dynamic scan over 2 hours for ⁸⁹Zr-DFO and ⁸⁹Zr-DFO-NPs. ⁸⁹Zr-DFO enters the interstitial parenchyma to a greater extent than ⁸⁹Zr-DFO-NPs and biodistribution at various timepoints from 3 hr to 12 days after Zr89-DFO-NPs in healthy mice (n=4 for 4 day, 7 day, 12 day, n=8 for 3 hr, 24 hr) (FIG. 4C). % total is calculated as activity in ROI over total injected activity Data as shown as the mean±/−s.d.; significant difference by two-tailed Mann-Whitney U test.

FIGS. 5A-5E. Zr-DFO-NPs accumulate preferentially in tumors. tumor-bearing mice were injected with ⁸⁹Zr-DFO-NPs and imaged continuously for 2 h. Pharmacokinetic curve of brain, spinal cord, and tumor site (FIG. 5A) Pharmacokinetic curve of cervical lymph nodes, bladder, and liver (FIG. 5B). Biodistribution at various timepoints from 6 h to 21 days after ⁸⁹Zr-DFO-NPs in tumor-bearing mice (n=4). % total is calculated as activity in ROI over total injected activity (FIG. 5C). Graph of bioluminescence intensity of brain and spinal cord tumor signal from mice (FIG. 5D). Graph of quantification of Cy5-NP area fraction in 10 representative sections of whole brain 24 hours after administration (n=5) (FIG. 5E), P=0.0005, one-way ANOVA

FIGS. 6A-6B are graphs showing the toxicity of BMN-673 free drug and BMN-NPs in mice). Tolerability of various doses of free BMN-673 and BMN-NPs in J:Nu mice was assessed by monitoring body weight and overall health conditions after a single IT treatment. The dose was administered to one mouse first, and if tolerated, was expanded to n=5. Red asterisk indicates animal died or was sacrificed due to excessive weight loss. Free BMN-673 was lethal at any dose at or higher than 0.06 mg/kg. Animals experienced weight loss less than 15% at 0.05 mg/kg. 0.03 mg/kg was determined as the maximum tolerated dose (MTD) (FIG. 6A). BMN-NPs was lethal at 1.25 mg/kg, but tolerated at lower doses without weight loss greater than 10%. (FIG. 6B) 0.5 mg/kg was determined as the MTD. J:Nu mice were treated with BMN-673 or BMN-NPs at the MTD, and complete blood cell counts, differential white blood cell counts, and platelet cell counts were performed to evaluate hematological toxicity. At 3 days post-treatment, free BMN-673 induced a decrease in all leukocytes except for monocytes, and also showed a reduction in platelet count. A lower level of decrease was seen for BMN-NPs across all cell counts. By 7 days post-treatment, BMN-NPs showed improvements in platelet, white blood cell, basophil, red blood cell count and hemoglobin conc. Free BMN-673 did not show an improvement outside of basophil count. For this study n=6 for each group.

FIGS. 7A-7F are graphs of mice with a tumor treated via a CM catheter, and treated with the same catheter via IT administration of either free BMN-673 (0.05 mg/kg one dose) or BMN-NPs (0.25 mg/kg one dose) (FIG. 7A). Two mice were removed from overall study due to no observable tumor growth. Survival curves for BMN-673 treated, BMN-NP treated, and control mice (n=6, one mouse removed from control and BMN free group for no tumor growth) (FIG. 7B). Change in body weights of all groups (FIG. 7C). Data presented as mean±/−s.d. Region-of-interest analysis of bioluminescence intensities from whole brain (FIG. 7D) Whole body bioluminescence images of DAOY tumor bearing mice. Bioluminescence scale for 1^{st} row is different from all remaining images. (NPs, FIG. 7E; Free drug, FIG. 7F).

### DETAILED DESCRIPTION OF THE INVENTION

### I. Definitions

“Nanoparticle,” as used herein, generally refers to a nanoparticle of any shape having a diameter from about 1 nm up to, but not including, about 1 micron, more preferably from about 5 nm to about 500 nm, most preferably from about 5 nm to about 100 nm. Nanoparticles having a spherical shape are generally referred to as “nanospheres”.

“Mean nanoparticle size,” as used herein, generally refers to the statistical mean nanoparticle size (diameter) of the nanoparticles in a population of nanoparticles. The diameter of an essentially spherical nanoparticle may be referred to as the physical or hydrodynamic diameter. The diameter of a non-spherical nanoparticle may refer preferentially to the hydrodynamic diameter. As used herein, the diameter of a non-spherical nanoparticle may refer to the largest linear distance between two points on the surface of the nanoparticle. Mean nanoparticle size can be measured using methods known in the art, such as dynamic light scattering.

“Monodisperse” and “homogeneous size distribution,” are used interchangeably herein and describe a plurality of nanoparticles where the nanoparticles have the same or nearly the same diameter or aerodynamic diameter. As used herein, a monodisperse distribution refers to nanoparticle distributions in which 80, 81, 82, 83, 84, 85, 86, 86, 88, 89, 90, 91, 92, 93, 94, 95% or greater of the distribution lies within 5% of the mass median diameter or aerodynamic diameter.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic” as used herein refers to substances that lack an affinity for water; tending to repel and not absorb water as well as not dissolve in or mix with water.

As used herein, an amphiphilic polymer is one which has one end formed of a hydrophilic polymer and one end formed of a hydrophobic polymer. As a result, when dispersed into a mixture of water and low water-solubility solvent such as many of the organic solvents, the hydrophilic end orients into the water and the hydrophobic end orients into the low water-solubility end.

Self-assembling refers to the use of amphiphilic polymers, alone or in mixture with hydrophilic and/or hydrophobic polymers, which orient in a mixture of aqueous and non-aqueous solvents to form nanoparticles, wherein the hydrophilic ends orient with the other hydrophilic ends and the hydrophobic ends orient with the other hydrophobic ends.

“Molecular weight,” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “therapeutic or prophylactic agent” refers to an agent that can be administered to prevent or treat one or more symptoms of a disease or disorder. Therapeutic agents can be a nucleic acid, a nucleic acid analog, a small molecule (less than 2000 D, less than 1500 D or less than 1000 D), a peptidomimetic, a protein, peptide, carbohydrate or sugar, lipid, or surfactant, or a combination thereof.

“Effective amount” or “therapeutically effective amount”, as used herein, refers to an amount of drug effective to alleviate, delay onset of, or prevent one or more symptoms of a disease or disorder. The terms “treating” or “preventing”, as used herein, can include preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

“Pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

“Biocompatible” and “biologically compatible,” as used herein, generally refer to materials that are, along with any metabolites or degradation products thereof, generally non-toxic to the recipient, and do not cause any significant adverse effects to the recipient. Generally, biocompatible materials are materials which do not elicit a significant inflammatory or immune response when administered to a patient.

“Biodegradable” as used herein means that the materials degrade or break down into its component subunits, or digestion, e.g., by a biochemical process, of the material into smaller (e.g., non-polymeric) subunits.

### II. Nanoparticle Formulations

### A. HPG-PLA Nanoparticles

The nanoparticles contain a core and a shell or coating. The shell is formed of hyperbranched polyglycerol (HPG). The HPG is covalently bound to hydrophobic polymer that form the core, such that the hydrophilic HPG is oriented towards the outside of the nanoparticles and the hydrophobic polymer is oriented to form the core.

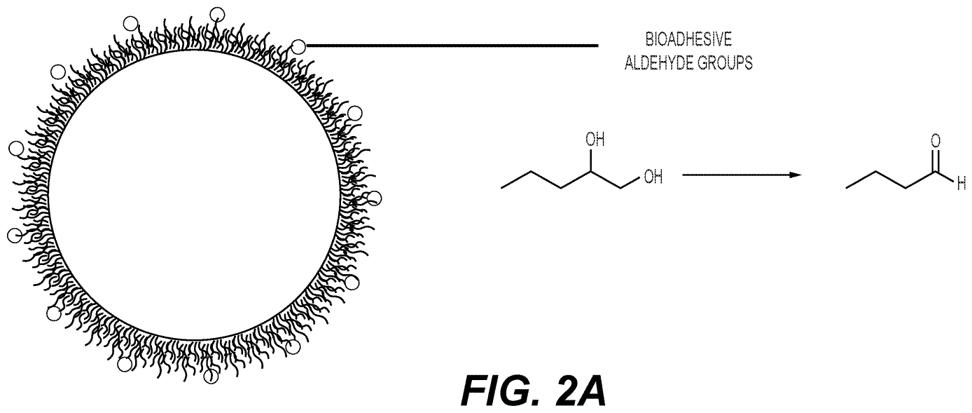

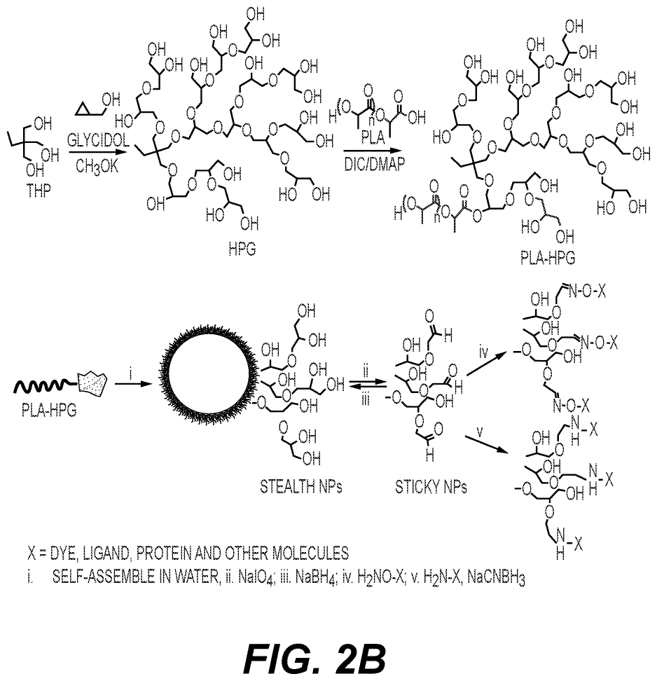

The HPG coating can be modified to adjust the properties of the nanoparticles. For example, unmodified HPG coatings impart stealth properties to the nanoparticles which resist non-specific protein absorption and are referred to as non-bioadhesive nanoparticles (NNPs). As used herein, the hydroxyl groups or other groups on the HPG coating are chemically modified to form functional groups that react with functional groups on tissue or otherwise interact with tissue to adhere the nanoparticles to the tissue, cells, or extracellular materials, such as proteins. Such functional groups include aldehydes, amines, oximes, and O-substituted oximes, most preferably aldehydes. Nanoparticles with an HPG coating chemically modified to form functional groups are referred to as bioadhesive nanoparticles (BNPs). The chemically modified HPG coating of BNPs forms a bioadhesive corona of the nanoparticle surrounding the hydrophobic polymer forming the core. See, for example, WO 2015/172149, WO 2015/172153, WO 2016/183209, and U.S. Published Applications 2017/0000737 and 2017/0266119. FIG. 2A is a cross-sectional schematic of a HPG NP and FIG. 2B is a schematic showing the synthesis of stealth nanoparticles and sticky nanoparticles.

The core of the NPs preferably is formed of polymers fabricated from polylactides (PLA) and copolymers of lactide and glycolide (PLGA). These have established commercial use in humans and have a long safety record (Jiang, et al., Adv. Drug Deliv. Rev., 57 (3): 391-410); Aguado and Lambert, Immunobiology, 184 (2-3): 113-25 (1992); Bramwell, et al., Adv. Drug Deliv. Rev., 57 (9): 1247-65 (2005)).

Hyperbranched polyglycerol (HPG) is a highly branched polyol containing a polyether scaffold. Hyperbranched polyglycerol can be prepared using techniques known in the art. It can be formed from controlled etherification of glycerol via cationic or anionic ring opening multi-branching polymerization of glycidol. For example, an initiator having multiple reactive sites is reacted with glycidol in the presence of a base to form hyperbranched polyglycerol (HPG). Suitable initiators include, but are not limited to, polyols, e.g., triols, tetraols, pentaols, or greater and polyamines, e.g., triamines, tetraamines, pentaamines, etc. In one embodiment, the initiator is 1,1,1-trihydroxymethyl propane (THP).

A formula for hyperbranched polyglycerol as described in EP 2754684 is

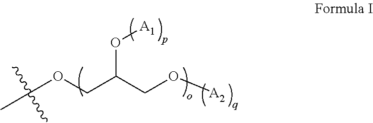

wherein o, p and q are independently integers from 1-100,
wherein A₁ and A₂ are independently

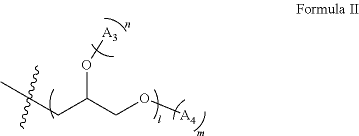

wherein 1, m and n are independently integers from 1-100.
wherein A₃ and A₄ are defined as A₁ and A₂, with the proviso that A₃ and A₄ are hydrogen, n and m are each 1 for terminal residues.

The surface properties of the HPG can be adjusted based on the chemistry of vicinal diols. For example, the surface properties can be tuned to provide stealth nanoparticles, i.e., nanoparticles that are not cleared by the MPS due to the presence of the hydroxyl groups; adhesive (sticky) nanoparticles, i.e., nanoparticles that adhere to the surface of tissues, for example, due to the presence of one or more reactive functional groups, such as aldehydes, amines, oxime, or O-substituted oxime that can be prepared from the vicinal hydroxyl moieties; or targeting by the introduction of one or more targeting moieties which can be conjugated directly or indirectly to the vicinal hydroxyl moieties. Indirectly refers to transformation of the hydroxy groups to reactive functional groups that can react with functional groups on molecules to be attached to the surface, such as active agents and/or targeting moieties, etc.

The hyperbranched nature of the polyglycerol allows for a much higher density of hydroxyl groups, reactive functional groups, and/or targeting moieties than obtained with linear polyethylene glycol. For example, the nanoparticles can have a density of surface functionality (e.g., hydroxyl groups, reactive functional groups, and/or targeting moieties) of at least about 1, 2, 3, 4, 5, 6, 7, or 8 groups/nm².

The molecular weight of the HPG can vary. For example, in those embodiments wherein the HPG is covalently attached to the materials or polymers that form the core, the molecular weight can vary depending on the molecular weight and/or hydrophobicity of the core materials. The molecular weight of the HPG is generally from between about 1,000 and about 1,000,000 Daltons, from about 1,000 to about 500,000 Daltons, from about 1,000 to about 250,000 Daltons, or from about 1,000 to about 100,000 Daltons. In those embodiments wherein the HPG is covalently bound to the core materials, the weight percent of HPG of the copolymer is from about 1% to about 50%, such as about 1, 2, 3, 4, 5, 10, 15, 20, 25, 30, 35, 40, 45 or 50%.

In some embodiments, the HPG is covalently coupled to a hydrophobic material or a more hydrophobic material, such as a polymer. Upon self-assembly, nanoparticles are formed containing a core containing the hydrophobic material and a shell or coating of HPG. HPG coupled to the polymer PLA is shown below:

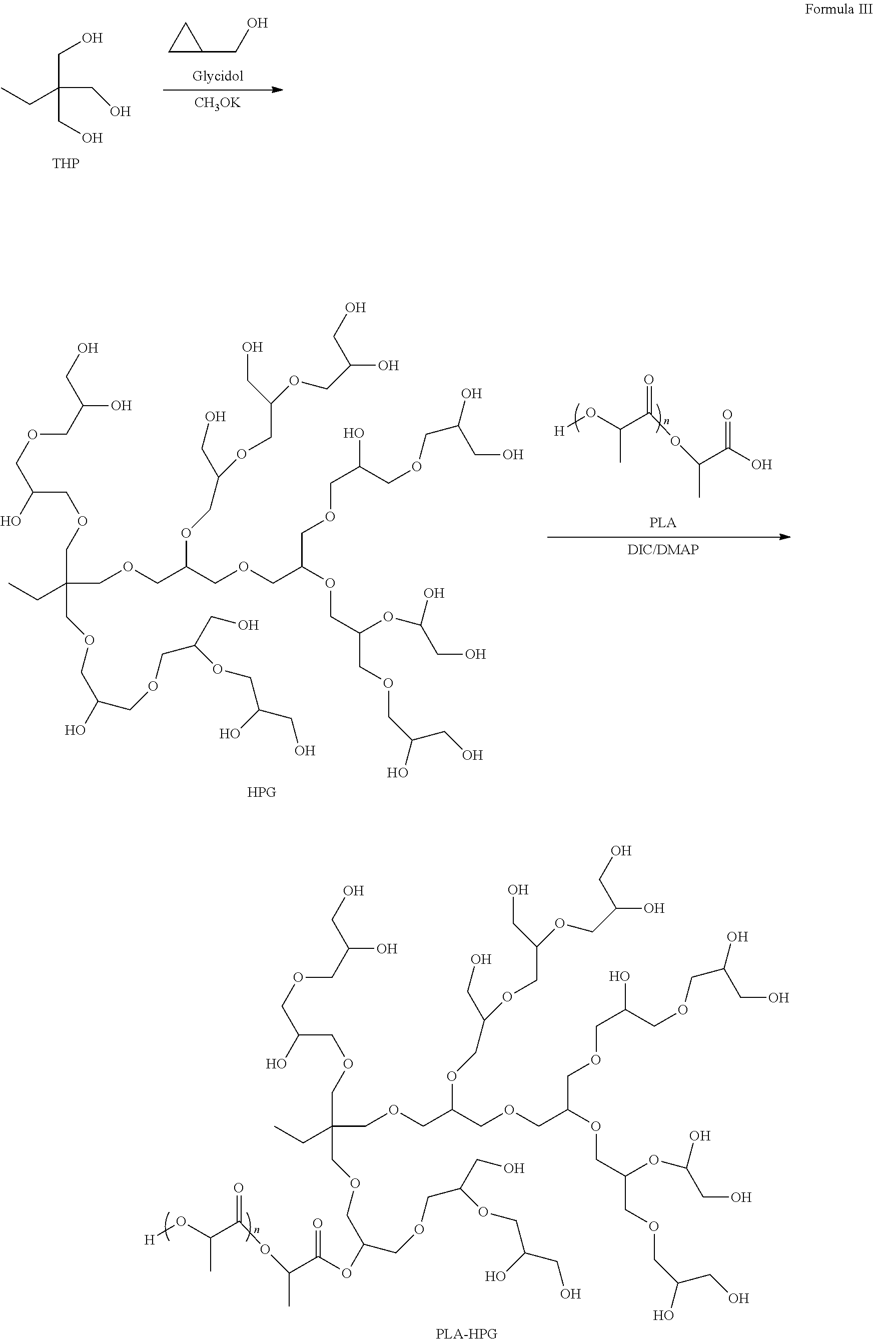

HPG-coated nanoparticles can be modified by covalently attaching PEG to the surface. This can be achieved by converting the vicinyl diol groups to aldehydes and then reacting the aldehydes with functional groups on PEG, such as aliphatic amines, aromatic amines, hydrazines and thiols. The linker has end groups such as aliphatic amines, hydrazines, thiols and O-substituted oxyamines. The bond inserted in the linker can be disulfide, orthoester and peptides sensitive to proteases.

PEG with a functional group or a linker can form a bond with aldehyde on PLA-HPG-CHO and reversed the bioadhesive state of PLA-HPG-CHO to stealth state. This bond or the linker is labile to pH change or high concentration of peptides, proteins and other biomolecules. After administration systematically or locally, the bond attaching the PEG to PLA-HPG-CHO can be reversed or cleaved to release the PEG in response to environment, and expose the bioadhesive PLA-HPG-CHO nanoparticles to the environment. Subsequently, the nanoparticles will interact with the tissue and attach the nanoparticles to the tissues or extracellular materials such as proteins. The environment can be acidic environment in tumors, reducing environment in tumors, protein rich environment in tissues.

HPG can be covalently bound to polymer that form the core of the nanoparticles using methodologies known in the art. For example, an HPG such as HPG can be covalently coupled to a polymer having carboxylic acid groups, such as PLA, PGA, or PLGA using DIC/DMAP.

The HPG can be initiated from hydroxyl, amine, and carboxylate terminated molecules, such as an alcohol with one or multiple long hydrophobic tail. In another example, the HP, such as HPG, can be initiated from special functionalized initiators to facilitate the conjugation to more materials. These special initiators include disulfide (Yeh et al., Langmuir. 24 (9): 4907-16 (2008)).

The HPG can be functionalized to introduce one or more reactive functional groups that alter the surface properties of the nanoparticles. The surface of the nanoparticles can further be modified with one or more targeting moieties or covalently bound to an HPG such as HPG via a coupling agent or spacer in organic such as dichloromethane (DCM), dimethylformamide (DMF), dimethyl sulfoxide (DMSO), tetrahydrofuran (THF), diisopropylcarbodiimide (DIC), 4-(N,N-dimethylamino)pyridine (DMAP), dicyclohexylcarbodiimide (DCC), DIC/DMAP, DCC/DMAP, Acylchloride/pyridine. In some embodiments, the polymer is functionalized/modified before nanoparticle formation.

HPG coated NPs can be transformed to aldehyde terminated NPs by NaIO₄ treatment (or carboxylic acid terminated by NaIO₄ treatment followed by sodium chlorite treatment) so the targeting moieties may be directly covalently attached to NPs via aldehyde (or carboxylic acid) groups on NPs and functional groups (amine, hydrazine, amino-oxy and their derivatives) on the targeting moieties or indirectly attached to the NPs via coupling agents or spacers (such as amino-oxy modified biotin and cysteine).

Because high molecular weight HPG has better resistance to non-specific adsorption to biomolecules, the low molecular weight components can be removed from the synthesized HPG by multiple solvent precipitations and dialysis.

In the preferred embodiment, a polyhydroxy acid such as PLA is selected as the hydrophobic core material because it is biodegradable and has a long history of clinical use. To covalently attach the PLA to HPG, the previous approach was to first functionalize the HPG with an amine and then conjugate the carboxylic group on PLA to the amine. This approach is efficient but cannot be used to make HPG as surface coatings since any amines that do not react with PLA will lead to a net positive charge on the neutral HPG surface and reduce the ability of HPG to resist adsorption of other molecules on the surface. To avoid this, a one-step esterification between PLA and HPG can be employed, which maintains the charge neutral state of the HPG. Alternatively, PLGA can be used as the hydrophobic core material for covalent attachment to HPG.

### B. Molecules to be Encapsulated or Attached to the Surface of the Nanoparticles

The nanoparticles may contain one or more types of molecules encapsulated within and/or attached to the surface of the nanoparticles. The molecules can be covalently or non-covalently associated with the nanoparticles. Molecules can be bound to the hydroxy groups on HPG before or after nanoparticle formation. Representative methodologies for conjugating molecules to the hydroxy groups on HPG are described below.

One useful protocol involves the “activation” of hydroxyl groups with carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix.

A useful coupling procedure for attaching ligands with free hydroxyl and carboxyl groups to polymers uses the cross-linking agent, divinylsulfone. This method is useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8.

Alternatively, the hydroxyl groups can be converted to reactive functional groups that can react with a reactive functional group on the molecule to be attached. For example, the hydroxyl groups on HPG can be converted to aldehydes, amines, or O-substituted oximes which can react with reactive functional groups on molecules to be attached. Such transformations can be done before or after nanoparticle formation.

Any suitable coupling method known to those skilled in the art for the coupling of ligands and polymers with double bonds, including the use of UV crosslinking, may be used for attachment of molecules to the polymer.

### C. Therapeutic, Prophylactic and Diagnostic Agents

Chermotherapeutic agents for treatment of brain tumors include Afinitor (Everolimus), Afinitor Disperz (Everolimus), Avastin (Bevacizumab), Belzutifan, Bevacizumab, BiCNU (Carmustine), Carmustine, Carmustine Implant, Danyelza (Naxitamab-gqgk), Everolimus, Gliadel Wafer (Carmustine Implant), Lomustine, Mvasi (Bevacizumab), Naxitamab-gqgk, Temodar (Temozolomide), Temozolomide, Welireg (Belzutifan) and Zirabev (Bevacizumab).

In a preferred embodiment, the agents to be delivered are radiosensitizers, most preferably PARP inhibitors (PARPi). Poly (ADP-ribose) polymerase-1 or PARP-1 is a multifunctional regulator of transcription, chromatin structure and genomic integrity activated by DNA breaks using NAD as a substrate.

A “PARP inhibitor” (PARPi) is an inhibitor of PARP-1, PARP-2, or PARP-3, though at this time, they are predominantly PARP-1 inhibitors.

Since radiation induce DNA damage in the DNA of all cells, selectively targeting PARP inhibitors to the cancer cells, rather than to the brain cells, increases the damage to the cancer cells at lower amounts of radiation than must be used if targeted to all cells, cancer cells as well as brain in the absence of the PARP inhibitors, which act as radiosensitizers. PARP binds to DNA, synthesizes PAR chains, which recruit repair enzymes, and repair DNA damage. PARPi inhibit repair by preventing base excision repair, so the single stranded break (SSB) in the DNA turns into a double stranded break (DSB).

Medulloblastoma are sensitive to PARPi in combination with radiation. For example, exposure of medulloblastoma cell line D283 to 1 micromolar Olaparib and radiation causes high levels of DSB, with the effect increases as a function of radiation strength. See van Vuurden, et al. Oncotarget 2:984-996 (2011).

Non-limiting examples of PARP inhibitors, that also can be classified as PARP-1 inhibitors, include olaparib, veliparib, CEP-8983 (II-methoxy-4,5,6,7-tetrahydro-IH-cyclopenta[a]pyrrolo[3,4-c]carbazole-I, 3 (2H)-dione) or a prodrug thereof (e.g. CEP-9722), rucaparib, E7016 (10-((4-hydroxypiperidin-I-yl)methyl) chromeno-[4,3,2-de]phthalazin-3 (2H)--one), INO-1001 (4-phenoxy-3-pyrrolidin-I-yl-5-sulfamoyl-benzoic acid), niraparib, talazoparib (BMN673), NU1025 (8-hydroxy-2-methylquinazolin-4 (3H)-one), 1,5-dihydroiso quinoline, 4-amino-1,8-naphthalimide, 2-nitro-6[5H] phenanthridinone, PD128763, and analogues, isosteres, and derivatives thereof (Curtin N J, et al. Therapeutic applications of PARP inhibitors: anticancer therapy and beyond. Molecular aspects of medicine 2013; 34:1217-56). Olaparib was one of the first PARPi to enter clinical trials for indications including breast, prostate and ovarian cancer patients carrying mutations in BRCA1 or BRCA2 genes, showing anti-tumour effect and side effects of grade 1 or 2 (Fong, Boss et al. 2009, N Engl J Med 361 (2): 123-134; Tutt, Robson et al. 2010, Lancet 376 (9737): 235-244).

### III. Methods of Treatment

### A. Patients to be Treated

The primary class of patients to be treated are children with cancers such as medulloblastomas and leptomenigeal tumors. However, the technology is suitable for use for treatment of any cancers of the CNS, and should be useful for delivery of other therapeutic, prophylactic or diagnostic agents for delivery to the CNS where release is desired over a period of up to a few days.

### B. Methods of Administering Nanoparticle Formulations

By encapsulating the drug in nanoparticles that are optimized for sustained drug release and retention in the CSF, one can release cytotoxic levels of drug in the CSF for prolonged periods to treat occult tumor cells.

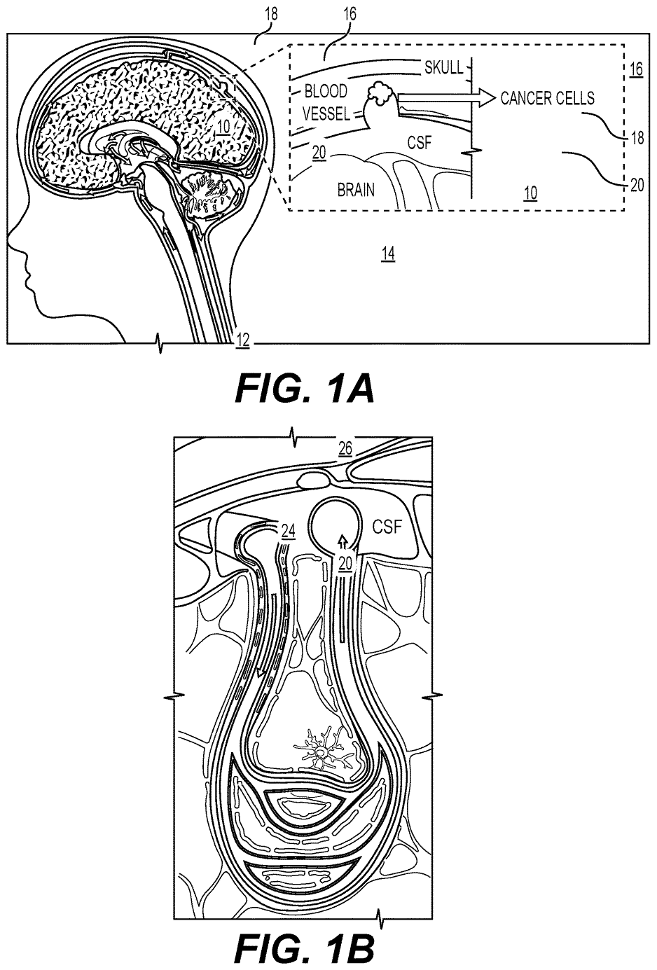

In intrathecal delivery, the drug is administered by injection into the subarachnoid space in the spinal column, using a local anesthetic. The drug then moves through the CSF into the CSF in the brain as shown in FIG. 1A.

As shown in FIG. 1A, the central nervous system includes the brain 10, the spinal cord 12, the cerebellum 14, the skull 16, blood vessels 18, and the cerebral spinal fluid (CSF) 20. As shown in FIG. 1B, drugs administered intrathecally travel via the CSF 20 in the periarterial space containing the arteries 24 and veins 26 and into the meningeal lymphatics 28 and arachnoid granulations 22.

In clinical settings, intrathecal delivery is used to treat patients with chronic pain by delivering opioids, or to treat leptomeningeal metastasis by delivering chemotherapy drugs. Despite intrathecal delivery of chemotherapy being the standard of care, patient median survival is limited to a few months. DEPOCYT®, an FDA approved sustained release lipid formulation of cytarabine, has been used in patients for leptomeningeal metastasis. However, these are “microparticles” and not NPs, and as such, they have limited diffusion and penetration characteristics. Thus, biodegradable and biocompatible drug-loaded NPs that can penetrate evenly through the CNS should confer an improvement to current treatment paradigms.

The present invention will be further understood with reference to the following non-limiting examples.

### Example 1: Nanoparticle Preparation

### Methods and Materials

### Blank PLA-HPG NP, Cy-5 NP, and Aldehyde-NP Synthesis

Blank PLA-HPG nanoparticles (NPs) were made with a single emulsion, solvent evaporation process (Deng, Y. et al. Biomaterials 35, 6595-6602 (2014)). Briefly, 50 mg of polymer was dissolved in 2.4 mL of ethyl acetate, and then dissolved in 0.6 mL of DMSO. This polymer solution was dropwise added to 4 mL of deionized water, sonicated, and then evaporated for 15 min on a rotary evaporator. NPs were then transferred to a centrifugal filter (Amicon Ultra-15, 100 kDa MWCO, Sigma Aldrich) and centrifuged three times at 4000×g to remove excess polymer and solvent. Nanoparticles were resuspended in deionized water (DI water) after the final spin and flash frozen in liquid nitrogen before being stored at −20 °C until usage. To prepare different sizes of NPs, different PLA-HPG co-polymers of various MWs were used.

To prepare Cy5-NPs, 50 mg of PLA-HPG polymer was combined with Poly (D,L) lactic acid-Cyanine 5 endcap (PolySciTech) at 10% (w/w) to and processed using the same process as before.

To prepare aldehyde-NPs, NPs were diluted to a concentration of 25 mg/mL, combined with equal quantities of 10×PBS and 0.1 M NaIO₄, and incubated on ice. After 20 min, one volume of 0.2 M Na₂SO₃ was added to quench the reaction. Aldehyde-NPs were then transferred to a centrifugal filter and washed three times at 4000×g.

### BMN-673 Loaded NP Synthesis

For BMN-673 loaded NPs, 20 mg of BMN-673 was used per 100 mg of PLA-HPG. 50 mg of polymer was dissolved in 1.2 mL of ethyl acetate, and 20 mg of drug dissolved in DMSO was added to the polymer solution. After this step, the BMN-673 NPs were prepared similarly to blank NPs. After multiple washes in a centrifugal filter with DI water, the NPs were filtered with a 1.5 μm glass microfiber filter (Whatman #6827-1315) before in vivo or in vitro use.

### Quantification of BMN-673 Loading

BMN-673 loading was calculated by creating 25 μL to 50 μL aliquots (triplicates) of the BMN-NPs and lyophilizing in a pre-weighed tube. Solutions were redissolved in DMSO, and then ran through an Agilent LC-MS 6120B (Agilent Technologies, Santa Clara, CA, USA) with standard curves to determine the loading of drug in NPs. The release of BMN-673 from the NPs formulations was measured for up to 7 days. NPs loaded with 5% w/w drug were dispersed at 50 mg/mL in either artificial CSF (aCSF) or PBS, and incubated at 37 °C. The suspension was centrifuged through a 3 kD filter at various time points (4 h, 8 h, 24 h, 48 h, 72 h, 5 day, 7 day. Filtrate was collected for HPLC analysis as previously described for BMN-673 loading, and the pellet was re-suspended in the same volume of PBS or aCSF for continued release.

### DFO-NP Synthesis

Aldehyde-NPs were conjugated to a chelator, deferoxamine mesylate (DFO, CAS #138-14-7, Sigma) to the nanoparticle surface, for further labeling with ⁸⁹Zr. Surface conjugation of DFO was achieved through reductive amination. After aldehyde conversion, NPs (25 mg/mL) were incubated with 1 molar equivalent of DFO mesylate for 4 h at room temperature. 40 molar equivalents of NaCNBH₃ were added, and NPs were incubated for an additional 40 h. DFO-conjugated NPs were washed four times to remove excess DFO mesylate and NaCNBH₃ and resuspended at a concentration of 25 mg/mL.

⁸⁹Zr-DFO-NP and ⁸⁹Zr-DFO Preparation

DFO-NPs were labeled for 30 min at room temperature with neutralized ⁸⁹Zr-oxalate in 0.25 M HEPES (pH 7.4) at a specific activity of μCi μg⁻¹. Radiochemical yield was evaluated using radio-thin liquid chromatography (radio-TLC). ⁸⁹Zr-DFO-NPs were washed three times using a centrifugal filter (Amicon Ultra-0.5, 100 kDa MWCO, Sigma Aldrich), and resuspended to a final concentration of approximately 40 μCi/μL (decay-corrected to the time of delivery). To determine uptake in the absence of NPs, ⁸⁹Zr-DFO was prepared; 0.1 mg/mL DFO mesylate was labeled with neutralized ⁸⁹Zr-oxalate in 0.25 M HEPES (pH 7.4) at a specific activity of 10 μCi μg⁻¹ and was incubated for 30 min at room temperature. Radiochemical yield was determined via radio-TLC. ⁸⁹Zr-DFO was then loaded into an activated SEP-PAK PLUS C18 cartridge (Waters Corp), washed twice with deionized water, and eluted with 95% ethanol. Excess ethanol was evaporated at 90 °C for 1 h, and ⁸⁹Zr-DFO was resuspended to a final concentration of approximately 40 μCi/μL.

To determine ⁸⁹Zr-DFO-NP stability, particles were incubated in 37 °C aCSF for 7 days. Each day, particles were spun down in a 100 kDa centrifugal filter, and activity measurements for both filtrate and retentate was taken using a Hidex AMG automatic gamma counter (Hidex, Turku, Finland).

### NP Characterization

All NPs were characterized with dynamic light scattering (hydrodynamic diameter, PDI, zeta potential) at a concentration of 5 mg/mL in DI water (Malvern, Zetasizer APS). For transmission electron microscopy, samples were prepared at 1.0 μg/mL, XYZ. Particle stability was measured using Malvern Nano-ZS in artificial cerebrospinal fluid (Harvard Apparatus, Holliston, MA, USA) at 37 °C with a standard operating procedure taking measurements every minute. A 100 ml solution of NPs was lyophilized in a pre-weighed Eppendorf tube to measure particle yield.

Xenografts and Cell lines

DAOY, D341, and D283 were purchased from the American Type Culture Collection (ATCC<Manassas, VA) and cultured following supplier instructions.

### Mice

All procedures were approved by the Yale University Institutional Animal Care and Use Committee and performed in accordance with the guidelines and policies of the Yale Animal Resource Center. BALB/C mice (Charles River, 8 weeks, female) or J:Nu mice (Jackson Labs, 8 weeks, female) were used in all studies unless otherwise indicated.

### Intracisterna Magna (ICM) Injection and Catheter Implantation

Mice were anesthetized with ketamine/xylazine i.p., and ophthalmic solution placed on the eyes to prevent drying and the head of the mouse while secured in a stereotaxic frame. After making a skin incision, the muscle layers were retracted and the cisterna magna exposed. For bolus injection, a Hamilton syringe (coupled to a 33-gauge needle) was used to deliver the volume of the desired solution into the CSF-filled cisterna magna compartment, the needle was left in place for 1-2 minutes to avoid backflow. Mice were then glued with Vet bond, sutured and allowed to recover on a heating pad until active.

Catheter implantation was done with 32G IT catheter (0046EO; ReCathCo). For mice implanted with catheters, the trimmed end of the catheter was inserted into the cisterna magna, fixed along the superficial lateral muscles with tissue glue (Histoacryl), and the outer muscle layers were sutured. The mouse was allowed to recover on a heating pad until active, and local anesthesia ointment was used on the wound. Animals were also given buprenorphine (0.06 mg/kg every 12 h) for pain management. For 3 d after surgery, injection of buprenorphine was repeated, and mice were monitored for any signs of pain and/or distress. For any administration with the catheter, a 27G needle was fitted to the outer catheter tube and used to deliver up to 6 μL of NP or drug.

### Immunohistochemistry

Cy5-NPs were dissolved at 50 mg/mL in DI water and 6 μL were delivered I.C.M in BALB/C female mice (n=5). At various time points, mice were imaged with in vivo bioimaging using the IVIS Lumina II In Vivo Imaging System (PerkinELmer) in vivo and ex vivo to confirm Cy5-NP signal. Whole body transcardiac perfusion with 10 mL of PBS and 10 mL 4% PFA was performed before tissue dissection. Tissues were fixed in 2% PFA overnight, left in 30% sucrose solution for 5 days before cryopreserved in Tissue-Plus OCT compound (Thermo Fisher Scientific). Tissues were cut into 20 μM sections coronally with a cryostat (Leica), collected onto gelatin-coated SUPERFROST® Plus slides (Thermo Fisher Scientific), DAPI stained, and kept at −20 C. Samples of mice dosed with 3 kD FITC-dextran and 3000 kD FITC-dextran were prepared in the same manner (n=5).

Images were acquired and stitched using EVOS microscope. For quantitative analysis with Fiji software, 10-15 representative brain sections were imaged using the wide-field microscope. Area fraction was assessed in all sections by dividing the area covered by labeled NPs over the area of the brain section.

### Toxicity Analysis

To investigate toxicity, healthy female nude mice were first administered a specific dose of either free BMN-673 or BMN-NPs ranging from 0.03 mg/kg to 1.25 mg/kg. The mice were monitored for weight-loss or other signs of distress, and if the dose was well tolerated, 4 additional mice were dosed and monitored (n=5).

For hematological analysis, healthy nude mice (n=5 per time point) were administered either free BMN-673 (0.05 mg/kg) or BMN-NPs (0.5 mg/kg). At 3 days and 7 days post administration, 100 μL of blood was drawn via cardiac puncture using 15 μL EDTA-coated tubes. Levels of blood cell counts were measured using HEMA VET® HV950 multispecies hematologic analyzer (Drew Scientific, Oxford, CT, USA). Spleens and livers were harvested, fixed in formalin overnight, and H&E stained for histopathological evaluation.

### Medulloblastoma Models

DAOY cells (ATCC) were inoculated in 8 week J:Nu female mice at 10\hat{ }5 cells suspended in 6 μL of DPBS. Tumor growth was followed by luminescence imaging twice a week, implantation. For luminescence imaging, mice were injected with 200 μL of a solution of D-Luciferin (15 mg/ml, Caliper Life Science) and imaged 5 minutes after injection using an IVIS200 in vivo imaging system (Xenogen, Caliper Life Science). An image showing light intensity (photons/second) was generated and the signal in each mouse was quantified using Living Image 4.0 software (Xenogen). On day 7 after tumor injection, all mice were randomized and treated with BMN-673 loaded NPs or free BMN-673 drug either once a week or bi-weekly for two weeks. Tumors were monitored for regression or progression with BLI, and left until neurological symptoms appeared or humane endpoint was reached (slow motion, >20% weight lose).

### Histology

Mice showing late-stage neurological brain tumor symptoms were sacrificed, and CNS tissues for histological analyses were collected, fixed overnight in formalin, and embedded in paraffin. The brain was then cut along the coronal plane and the spinal cord was transversally sectioned according to cervical, thoracic, lumbar, and sacral regions. The location and extent of primary tumor and associated metastases was analyzed by standard H&E stains.

### PET Imaging

For initial studies of biodistribution ⁸⁹Zr-DFO-NPs (50 μCi), or ⁸⁹Zr-DFO (50 μCi) were administered i.e.m to BALB/C female mice (n=2), which were immediately scanned on an Inveon PET/CT scanner (Siemens Healthcare Global) under an isoflurane/oxygen gas mixture (2% for induction and maintenance) for 120 min dynamic scan. CT images were acquired in addition to PET for anatomical delineation. Half-life was determined with a XYZ statistical analysis on Prism.

For long-term studies in healthy mice, ⁸⁹Zr-DFO-NPs (150 μCi) were administered i.e.m (n=4) and 3 hrs later, PET scanned for 10 minutes. CT images were acquired in addition to PET for anatomical delineation. PET scans were repeated on subsequent days (24 hr, 4 day, 7 day, 12 day) with scan durations of 10-60 minute.

For studies in tumor-bearing mice, xenografts were generated with DAOY cells as described above. 3 cohorts of n=2 mice with visible tumors via IVIS imaging in both the brain and spinal cord were selected, and ⁸⁹Zr-DFO-NPs (200 mCi) were administered i.e.m via a catheter. The first cohort was PET imaged for a continuous 120 min dynamic scan. For the remaining cohorts, PET and CT images were acquired at 6 hr, 24 hr, 4 day, 7 day, 12 day, and 21 days.

All images were reconstructed using an ordered subset expectation maximization (OSEM-3D) algorithm and analyzed using Inveon Research software (Siemens Healthcare Global). Regions of interest (ROI) were manually defined on the CT image for the following regions: brain, spinal cord, all lymph nodes, liver, spleen, heart, lungs, stomach, kidneys, bladder, bones. Radioactivity concentration within the region of interest is reported in units of Bq/mL, which was converted to standardized uptake value (SUV) mean and % injected dose/gram.

After the final PET/CT scan, animals were euthanized, and tissues of interest (brain, spinal column, liver, kidney, spleen, lungs, muscle, heart, bone, stomach, cervical lymph nodes, peripheral lymph nodes) were collected, blotted, and weighted. Radioactivity was measured by gamma counting and normalized as Bq/gram.

### Statistical Analysis

Data analysis and visualization was performed using Prism 7.0 (GraphPad Software). Graphs represent either group mean values±s.d. (as indicated in the figure legends) or individual values. P values were calculated with log-rank statistics for survival analyses, then repeated-measures analysis of variance (ANOVA). A one-way ANOVA was used for multiple comparisons, and a Mann-Whitney U test was used for comparison between two groups. P<0.05 was considered statistically significant. P values are denoted with asterisks: P>0.05, NS; *P<0.05; **P<0.01; ***P<0.001; and ****P<0.0001

### Results

### Engineering NPs for Optimal CNS Retention

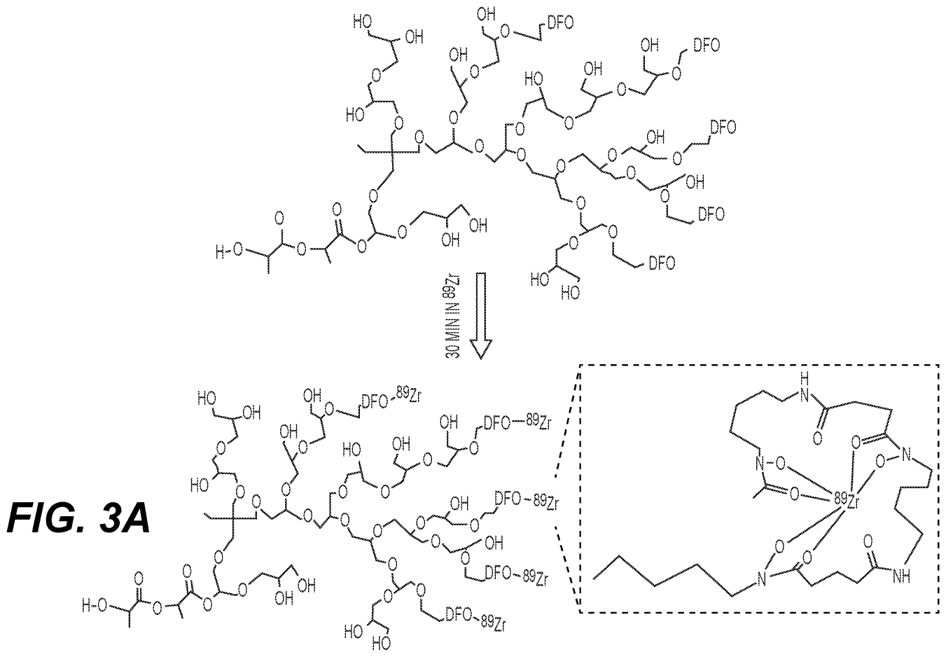

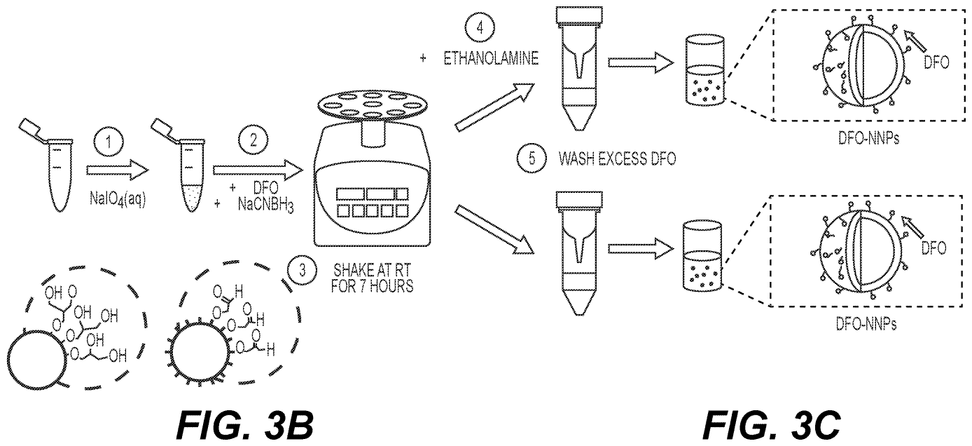

Particle size and surface charge were determined for a range of particle compositions (FIG. 3A-3C). To synthesize covalently tethered fluorescent dye nanoparticles for effective tracking via microscopy, NPs were produced with blends of PLA-HPG copolymer and a 5% Cy5-PLA conjugate. Unlike dyes, which may leak from NPs and complicate interpretation of measurements, Cy5-PLA conjugation ensures that fluorescent signal originates from the NPs themselves. It was hypothesized that PLA-HPG NPs would similarly exhibit clear advantages in the CSF environment after direct CSF injection via the cisterna magna (CM). Cy5-PLA-HPG NPs (Cy5-NPs) was administered to healthy mice and prepared frozen brain and spinal cord sections 24 h and 48 h post-injection. At 48 h, significant accumulation of Cy5-NPs was detected in the leptomeninges and perivascular spaces, without parenchymal uptake in all coronal sections of the brain. An even deposition of Cy5-NPs surrounding the outer layer of the spinal cord was also administered.

CNS retention of three distinct sizes of PLA-HPG NPs: 90 nm, 150 nm, and 210 nm was assessed six hours after intra-CSF delivery via the cisterna magna, mouse brain and spinal cord sections by imaging. FITC-conjugated dextran (either M_{w}=3,000 or 3,000,000) served as size-fractionated controls (diameter estimated to be <4 nm and >60 nm respectively). Although all mice administered NPs showed retention in the brain and spinal cord leptomeninges, the 90 nm NPs covered the greatest % of area in fresh frozen coronal brain sections as quantified by fluorescent signal. In comparison, while a strong fluorescent signal was detected for both dextrans at 30 min after injection, no signal was detected in any brain or spinal cord sections of mice that were dosed with 3 kD and 3000 kD dextran 6 hours post-injection.

Next, the effect of surface charge and surface chemistry of the PLA-HPG NPs was assessed. HPG on the NP surface was converted into an aldehyde-rich corona with enhanced bioadhesive properties (termed aldehyde-NPs). The conversion of the vicinal diols on the HPG coating to aldehydes is produced by brief sodium periodate treatment, and monitored by the change in zeta potential, which is lower for aldehyde-NPs. Despite the fact that surface charge and chemistry has a significant effect on plasma half-life and organ accumulation after i.v. or i.p. delivery, they had a negligible effect on CSF distribution in the leptomeningeal region. No meaningful differences were detected in CNS retention between the PLA-HPG aldehyde-NPs and PLA-HPG NPs over 7 days of ex vivo whole-body imaging with the XENOGEN® in vivo imaging system (IVIS). Both formulations of NPs were well retained in tumor-free brain and spinal cord of healthy mice at all timepoints examined with no difference in fluorescent radiance flux as measured in vivo and ex vivo. In case there were differences in accumulation along lymphatic clearance pathways, the % of Cy5-NP positive cells and % of Cy5-NP-aldehyde positive cells in the mandibular and deep cervical lymph nodes of tumor-free mice and tumor-bearing mice was assessed. No meaningful differences in accumulation of NPs in macrophages (CD11b+CD11c−) and dendritic cells in the lymph nodes based on surface chemistry was detected.

NPs with the most optimal characteristics for CNS retention exhibited a spherical morphology under electron microscope analysis, with an average hydrodynamic diameter of 90-100 nm. The zeta potential of the NPs used in the remainder of the studies averaged around-10 mV.

### NPs Show Higher CNS Retention Compared to Small Molecules.

Studies were conducted to clarify the detailed in vivo distribution kinetics of the NPs in the CSF space. To image the NPs with PET, a NP PET probe with analogous properties was developed. Positron-emitting zirconium (⁸⁹Zr) was used to label aldehyde-NPs, due to its stability and long half-life (72 h), and availability of a well-characterized chelating agent, deferoxamine (DFO) for ⁸⁹Zr. The aldehyde-NP surface was first functionalized with DFO-mesylate via a Schiff-base reaction, and no change in hydrodynamic diameter was detected, but a modest change in zeta-potential from −10 mV to −5 mV was observed. The DFO-grafted aldehyde-NPs were subsequently used to chelate ⁸⁹Zr by a 30 min incubation. After examining the stability of the complex with radio-thin layer chromatography, the resulting ⁸⁹Zr-DFO-NPs were washed extensively in a 300 kD filter tube to remove any unconjugated ⁸⁹Zr or ⁸⁹Zr-DFO from the surface of the aldehyde-NPs. A stability test in artificial CSF (aCSF) at 37 °C showed no loss in ⁸⁹Zr from the NP surface over 7 days.

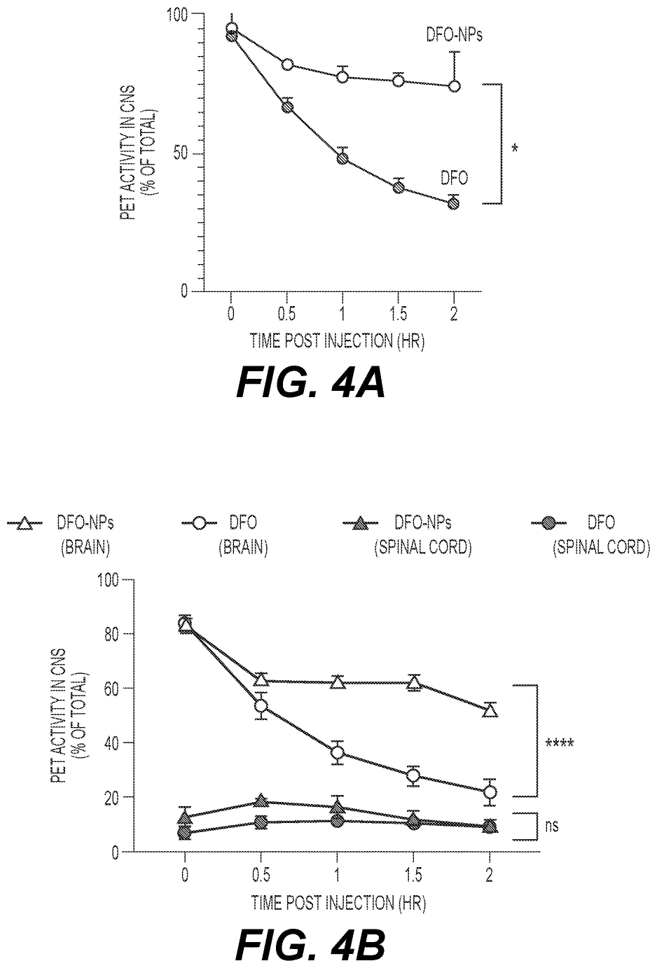

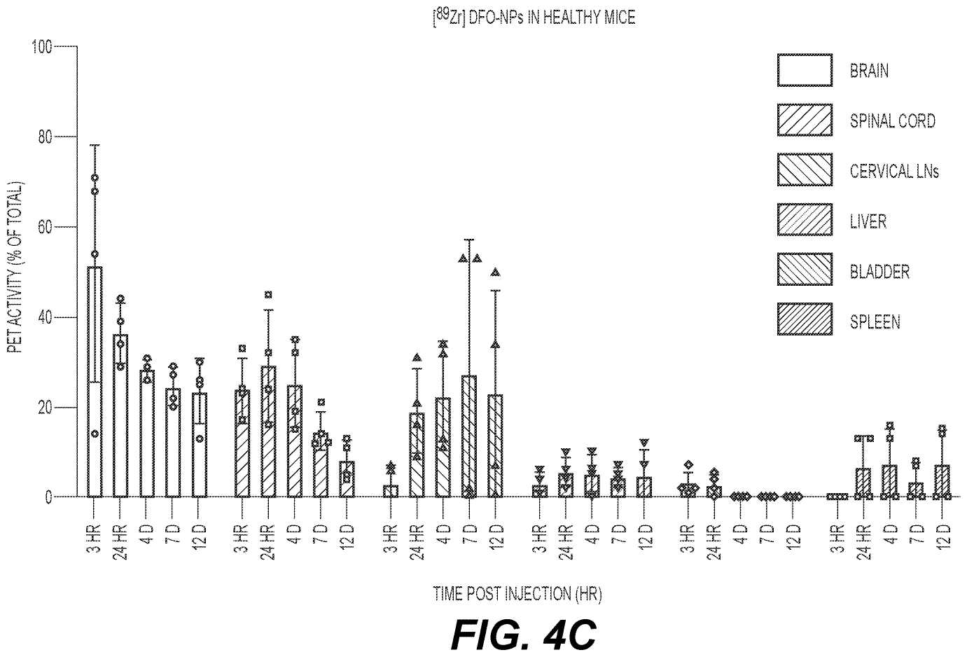

The quantitative biodistribution of the NPs into all major organs over time was measured. A 2 h continuous scan was conducted to determine the distribution and timing of NP after IT administration, in mice without tumors, using free ⁸⁹Zr-DFO as a control. Within 5 min of injection, indicated as the 0 h time point, NPs were observed distributing from the site of injection (CM) into the brain and cervical regions of the spinal cord (FIG. 4A, 4B). The level of ⁸⁹Zr-NP recorded in the CNS dropped slightly and then remained constant for the next 2 h, with limited distribution of signal to the systemic circulation or other organs. In contrast, ⁸⁹Zr-DFO distributed immediately throughout the subarachnoid space, similar to the NPs, but then distributed from the CNS into systemic circulation, with less than 30% of the PET signal detectable in the CNS by 2 h. The ⁸⁹Zr-DFO had a CNS half-life of ~60 min, which is comparable to known half-lives of small molecules following IT delivery. In comparison, the levels of ⁸⁹Zr-DFO-NPs were relatively stable in the brain and spinal cord during the first 2 hr. Additionally, ⁸⁹Zr-DFO-NPs showed differences in brain distribution patterns, with higher accumulation in the olfactory bulb than ⁸⁹Zr-DFO as seen by the more defined shape of the signal (FIG. 4C), which is related to the size difference between the two materials (100 nm vs <1 nm).

An accumulation of PET signal in the bladder a short time after NP delivery, indicating that some ⁸⁹Zr was reaching the bladder. This bladder signal is unlikely to be due to extravasated NPs. The size limitation of the kidney glomerular filtration is 48 kDa, or 5 nm for polymers such as PEG and dextran, and it is improbable that NPs degrade rapidly enough in the first 5 min after delivery to accumulate in the bladder. A comparable immediate spike and fall in bladder signal after IT delivery of DFO-⁸⁹Zr was observed. Free ⁸⁹Zr accumulates in bones and joints. It was confirmed that there is no significant level of free ⁸⁹Zr in the NP preparation. In subsequent studies, no signal was observed in the heart, lungs, joints, or muscle at any time points, and no signal in the kidneys and bladder was observed after the first 24 hours, indicating rapid clearance of ⁸⁹Zr-DFO from the renal system.

Next, ⁸⁹Zr-DFO-NPs IT was administered to animals without tumors and monitored the radioactivity signal over 12 d with serial PET/CT at predetermined time points (n=4). At 3 h, it was found that at least 60% of the signal was in the brain, and at least 20% of the signal was in the spinal cord (<80% in the CNS). The degree of activity in the brain gradually decreased over the first 4 d, eventually reaching 30%. This level of activity was observed in the brain for at least 12 d, the last day of imaging. In the spinal region, the decline in activity was slower over the first 4 d, with a similar level of activity observed at these time points, about 25%. On day 7, and 12, there was a decrease in signal to ~20% and ~10%. Ex vivo gamma counting after 12 d confirmed the in vivo imaging findings, with the brain exhibiting the highest Bq/g signal (44% of total mass-normalized Bq activity), then the liver (16%), spleen (34%), and cervical lymph nodes (3%). No identifiable signal was detected in the heart, GI organs, kidneys, and bladder.

### NPs Show Higher CNS Retention in Xenograft Tumor Model

CNS retention of ⁸⁹Zr-DFO-NPs was assessed in tumor-bearing mice. A leptomeningeal metastatic medulloblastoma model was administered DAOY cells stably expressing luciferase by IT injection into the CM. By day 14, BLI showed tumor growth throughout the CNS, including widely disseminated tumors in both the cerebellum and spinal cord. To establish that the presence of tumors did not result in BBB breakdown, mice were injected with Gd-DTPA MR contrast agent intravenously and imaged with MRI to confirm no BBB penetration occurred as compared to control. Two mice were injected intravenously with Gd-DTPA, and imaged via MRI after 20 minutes. There was no detectable Gd-DTPA signal in the brain, confirming an intact BBB. However, acute hydrocephalus was observed in both mice, most likely due to tumor obstruction of CSF flow. H&E images of the brain revealed a high tumor burden in the brain cerebellum, ventricles, and in the spinal leptomeninges.

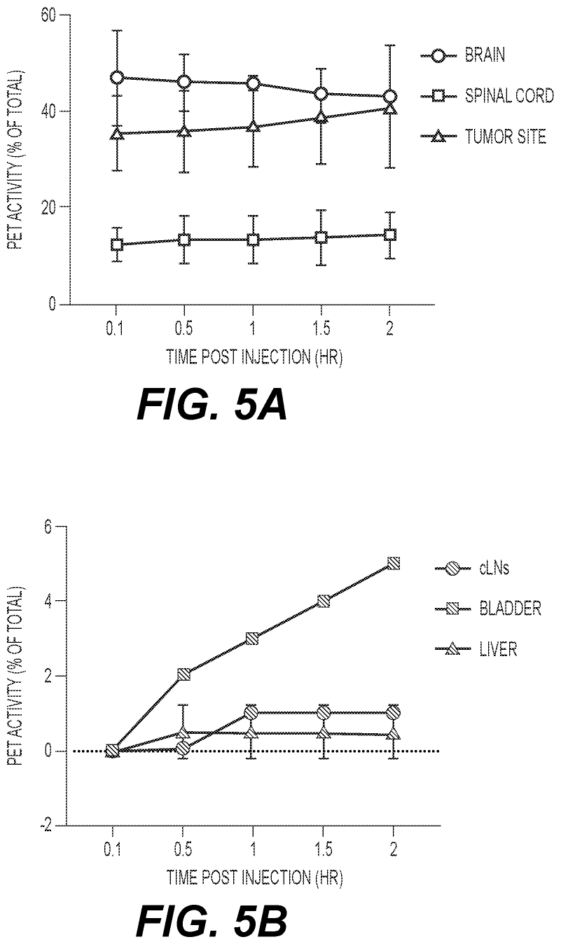

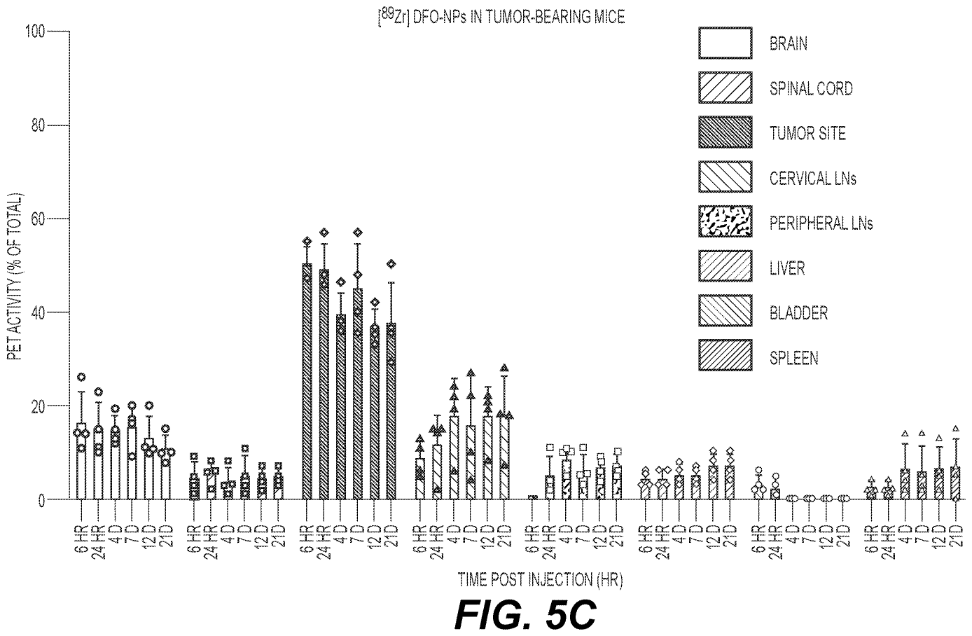

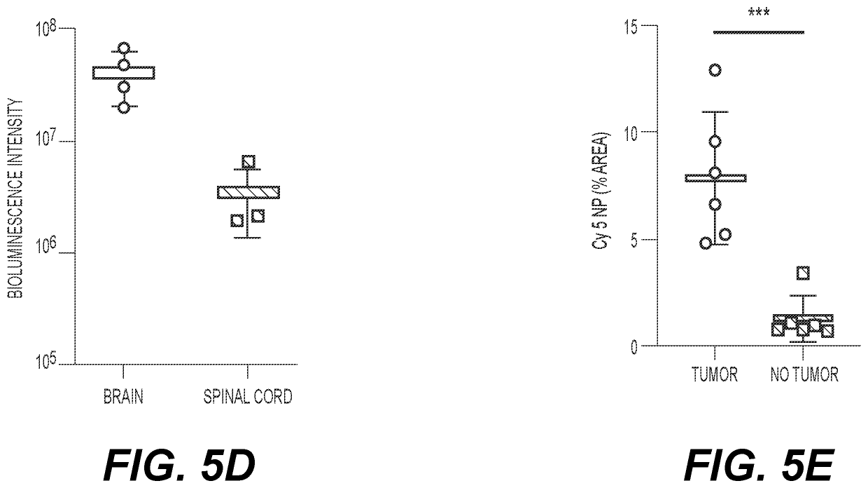

The biodistribution and uptake of ⁸⁹Zr-DFO-NPs in tumors and normal tissue was evaluated throughout a 21-day period using PET/CT imaging in two cohorts of mice (FIGS. 5A-5E). The first cohort (n=2) was scanned continuously for 120 min after administration. Within the first 5 min, 30% of the total injected dose was detected in the tumor, which gradually increased to 40% over the next 2 h. The next cohort (n=4) were scanned at predefined intervals (6 h, 24 h, 4 d, 7 d, 12 d, 21 d). There was significantly more uptake in the tumor than in any other normal tissue, with 50% of total injected dose identified in the tumor site, which was in the cerebellum. Over the next 21 d, levels in the brain and spinal cord remained stable at roughly 15% and 10% of total activity, respectively. Higher levels of peripheral organ accumulation was observed with the tumor bearing mice compared to healthy mice, with accumulation in the cervical lymph nodes, peripheral lymph nodes, liver, and spleen. At no point did any of these levels exceed 20% of overall activity. In general, tumor bearing mice showed longer retention of NPs in the CNS, most likely due to uptake of NPs by tumor cells. At day 21, 40% of PET activity remained detectable in the tumor site, which could be visualized using maximum intensity projections of the PET activity.

NP accumulation was assessed at the cellular level by administering Cy5-NPs in tumor-bearing mice and observing via microscopy. Dense NP accumulation and uptake in areas of tumor 7 d after injection was detected. NP retention in this area of the cerebellum was not observed in healthy mice. There was also a lower density of NP accumulation in the leptomeninges of the brain and spinal cord than in healthy animals, indicating that the NPs circulate through the perivascular space in a similar manner, but accumulate at significantly higher density at sites with tumor than in healthy brain or spinal tissue.

The presence of Cy5-NPs in areas of parenchymal tumor, and the accumulation of NPs in the deep and superficial cervical LNs as detected by PET raised the possibility of several transport pathways of the NPs into the tumor microenvironment and to the cervical LNs. NP accumulation at tumor sites was aided by the presence of immune cells. Cy5-NPs were injected into mice with tumors, and after 48 h, collected and sectioned the brain before staining for F4/80 (a marker for macrophages) and Iba1 (a marker for microglia). At 48 h, the presence of activated microglia (red) and tumor associated macrophages (green) was detected within the tumor bulk and detected significant colocalization of NPs with both cell types. In WT mice without tumors, no NP accumulation in the brain parenchyma was observed, only in CSF-bathed regions such as the choroid plexus, without any microglia or macrophage association. These results indicate that NP penetration and transport into the tumor bulk is aided by tumor-associated immune cells. A portion of NPs in the tumor microenvironment were not associated with either macrophages or microglia. These NPs could have been taken up by medulloblastoma tumor cells specifically, or by other cells in the brain parenchyma such as astrocytes.

To further examine the pathway in which NPs are cleared from the CNS, the meninges of the tumor-bearing mice were isolated at 48 h after ICM injection of Cy5-NPs, and stained for either Lyve-1 and CD45, or Lyve-1 and CD31. NP accumulation occurred throughout the entire meninges, but particularly along the sinuses (with higher density in the transverse sinus over the superior sagittal sinus). Close observation of the lymphatics and blood vessels of the transverse sinuses revealed that NP accumulation co-localized with regions of Lyve-1 and CD31 staining, but there are clusters of NPs that are neither associated with meningeal lymphatic vessels nor blood vessels. When looking at CD45 stained transverse sinus, it was found that NP accumulation frequently, but not always, co-localized with regions of Lyve-1 and CD45 immune cell staining.

### Polymeric Encapsulation of BMN-673 Alters the Toxicity Profile of Drug in Animal Studies

As a proof-of-concept, it was determined whether the PK properties and activity of a new class of drugs, PARPi, could be improved. BMN-673 was selected for its potent PARP trapping properties and ability to induce toxicity at very low doses. BMN-673 loading in NPs varied from 1% to 5% (w/w) depending on the solvent ratios and drug to polymer ratios used during NP preparation. The NPs without the aldehyde-modified surface were selected due to their similar persistence in the brain. The BMN-673 release rate from these NPs was similar in both CSF and PBS at 37 °C, averaging 60% release over 3 days. The relative cytotoxic activity of free BMN and BMN-NPs were determined on 3 MB cell lines: DAOY, D341, and D283. While both agents were cytotoxic at a range of 10 nM to 1 μM, the NPs were more potent with a lower IC50 value.

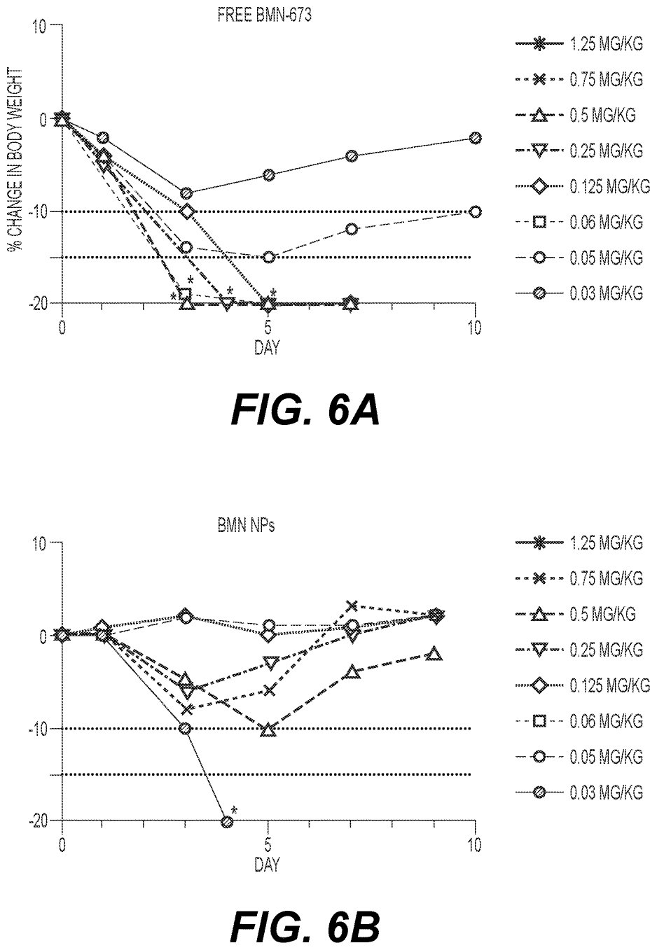

Toxicology studies were conducted in healthy female nude mice to characterize the safety of BMN-NPs in vivo. The maximum tolerated dose (MTD) were determined for single IT dosing of BMN NPs or free BMN in nude mice (FIGS. 6A-6B). Increasing doses of either BMN-NP or free BMN-673 were administered to animals. Body weight loss and overall animal health were closely monitored. MTD values for BMN NPs were tenfold higher than for free BMN. The median lethal dose for IT administration of free BMN-673 was 0.125 mg/kg. Animals treated with greater than 0.06 mg/kg developed a variety of toxicity-related symptoms, including lethargy, labored breathing and, in some cases, death. The MTD (with a single dose) was determined to be 0.05 mg/kg. A slightly lower dose, 0.03 mg/kg twice a week was tolerated with less than 10% body weight loss. By comparison, BMN-NPs were well tolerated at all doses tested at or under 0.5 mg/kg, which was the maximum dose allowed in one infusion due to IT volume dosing limits. To achieve IT doses of more than 0.5 mg/kg, the mice were dosed multiple times in the same day, within 3 h, and the lethal dose of 1.25 mg/kg was determined. At 1.25 mg/kg, there was delay in the onset of acute toxicity symptoms, which is presumably due to delayed drug release from the NPs.

To further test systemic toxicity differences between BMN-NPs and free BMN, red blood cells, white blood cells, and platelet levels were monitored in mice at day 3 and day 7 following two bi-weekly administrations at MTD (FIG. 5C). Mice treated with free BMN at 0.05 mg/kg/week had progressive leukopenia and thrombocytopenia at day 3 which did not improve appreciably at day 7. NPs dosed tenfold higher, at 0.5 mg/kg/week, maintained considerably more normal WBC, RBC, and PLT levels over the course of treatment, and all cell counts except for eosinophils returned to normal levels by day 7.

### BMN-673 NPs Show Superior Activity Compared to Free BMN-673 in Xenograft Tumor Model

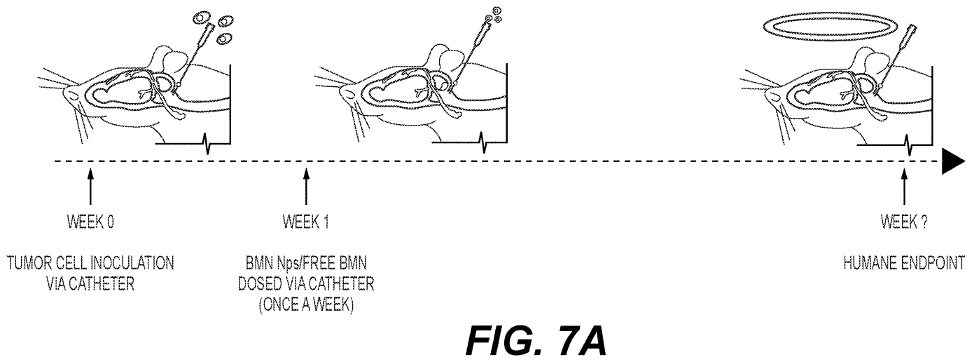

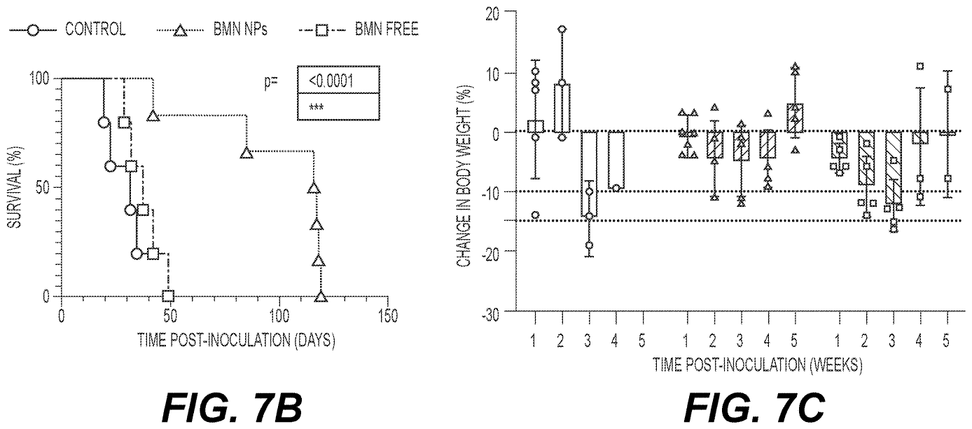

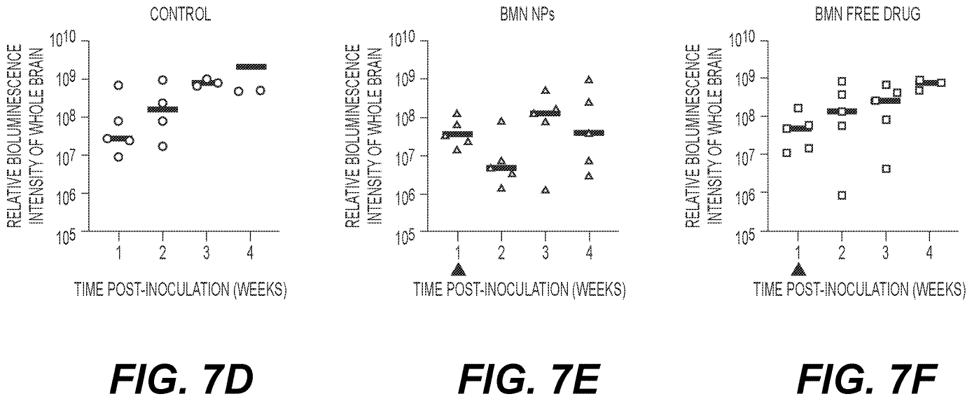

The improved therapeutic index of BMN-NPs over BMN demonstrated improved effectiveness in vivo tumor xenograft models. Intra-cisternal transplantation of DAOY cells stably expressing luciferase was used for in vivo studies. A surgical catheter was implanted into their cisterna magna, and used for both cellular implantation and IT dosage during treatment. Mice were treated when their tumoral luminescence burden was detectable at 105 BLI (unit), 7 days post-implantation. Mice were treated once, at the same dose level of 0.1 mg/kg with either BMN NPs or free BMN-673 (FIG. 7A). BMN-NP-treated tumors grew at a substantially slower rate than BMN-673 (free drug) treated tumors. The week after the dose, resulted in the largest reduction in tumor BLI, resulting in delayed growth in subsequent weeks that was not observed in the free BMN-673 group. Despite the fact that the dose was more than the MTD for free BMN-673, a tumor reduction benefit was observed in only one out five mice. Furthermore, the free BMN-673-treated mice lost significantly more weight than the BMN-NP group (FIG. 7C). Consistent with the BLI findings, mice treated with BMN-NPs lived significantly longer than those treated with free drug alone, with enhanced median survival of 56 days (FIG. 7B).

This study was repeated using twice-weekly dosing of 0.25 mg/kg of BMN-NPs or 0.03 mg/kg for free BMN-673 (FIGS. 7D-7F). These equitoxic doses are equivalent to roughly 50% of MTD, and similar levels of mean weight loss was observed in both treatment groups. A more dramatic tumor regression was observed in the NP treated group. The NP treatment had a substantial antitumoral impact in all the mice, especially one week after the doses were administered. BMN NPs induced complete regression in several treated animals, while tumors continued to progress with treatment of free drug. In NP-treated mice where the tumor progressed, a median survival benefit of 5 weeks was compared to the free drug group, and a survival benefit of 6 weeks compared to the control group. In this study, median survival in all treatment groups generally reflected the tumor growth rates evaluated by bioluminescence, suggesting that the mice succumb to cancer rather than drug-induced side effects. In addition, a significant improvement in rates of metastasis formation in the leptomeninges was measured by bioluminescence imaging in the treatment groups. BMN-NPs, and to a lesser degree, BMN-673 free drug, significantly reduced the incidence of CNS metastases. In comparison, over 80% of untreated animals developed evident spinal cord dissemination and required euthanasia around week 4, due to severe hydrocephalus.

BMN-673 NPS Synergize with Temozolomide when Given in Conjunction in a Xenograft Model

To demonstrate sensitivity of the medulloblastoma cell lines to TMZ and BMN-673 synergy, DAOY, D341, and D283 cells were treated with free BMN and BMN-NPs and performed conventional IC50 analyses. When combinatorial indexes were calculated using these two drugs, cell treated with incremental log-fold increases in BMN-673 in the presence of TMZ demonstrated decreased viability as quantified with the CellTiter-Glo viability assay compared to cells treated with BMN-673 alone. When BMN-673 and TMZ combinatorial index values were calculated using a classical Loewe synergy model, there was a high levels of synergy. Drug interaction assays with other commonly used, brain penetrant chemotherapy agents (Lomustine, Topotecan, Irinotecan, Cyclophosphamide) did not show consistent synergist action with BMN-673 across all cell lines.

Synergy was observed in vitro using a D341 xenograft model, where the cells were inoculated in a manner identical to the DAOY cell line. In the D341 model, most mice develop spinal metastasis that is detectable by IVIS imaging within 7 days of inoculation, so it was tested whether BMN-NPs and free BMN were efficacious against existing spinal metastasis by starting treatment at day 7 in the presence of leptomeningeal metastasis. Four repeat doses of BMN-NPs were efficacious in reducing cerebral and spinal tumor burden in the D341 model, whereas the free BMN-673 treatment (at an equitoxic dose to the NPs) showed no effect. This therapy was improved with a temozolomide (TMZ) combination approach. The synergy between TMZ and PARPi is well documented in vitro, as PARP1 trapping sensitizes cells to the DNA methylation mechanism of TMZ, but in vivo, a reduction in the PARPi dose is necessary to prevent acute toxicity. The BMN-NP and TMZ combination was well-tolerated in mice without a necessary dose-reduction and led to tumor clearance in 4 out of 6 mice tested. The combination of free BMN-673 and TMZ was not tolerated at even the lowest doses of BMN-673.

Overall, these results indicate that intra-CSF delivery of nanoparticle encapsulated drugs is a viable treatment regimen for MB with a meaningful treatment advantage over intrathecal administration of free drug alone in terms of pronounced and sustained in vivo activity.

### DISCUSSION

Direct infusion of drugs into the CSF has emerged as a promising method of circumventing the BBB in the treatment of medulloblastoma. Given that leptomeningeal recurrence remains the leading cause of mortality in patients, there is compelling rationale for a delivery route that increases intra-CSF drug exposure. Despite the potential advantages of intra-CSF delivery, maintaining high levels of drug concentration in the subarachnoid space against the rapid CSF convective flow and clearance has remained a steady challenge. The results described herein use a PLA-HPG NP platform that remains in the subarachnoid region for long periods of times, in contrast to the fate of freely administered small molecules. Long-term retention of the NPs in the CSF space of tumor-free mice, as well as preferential accumulation and retention in CSF-adjacent tumors, was demonstrated using PET/CT and fluorescent whole-body imaging. Increasing accumulation of nanoparticles in the tumor early-on in circulation lessens the probability of mononuclear phagocytic system and renal system clearance.

The vast majority of polymeric NPs exhibit substantial absorption in the spleen and clearance organs such as the liver and kidney, potentially limiting their therapeutic use. Even with functionalized BBB penetrating modalities, NP accumulation in the brain when delivered i.v is typically limited to less than 1% of total activity, and bulk of the delivered NPs are processed by the spleen and liver. HPG coated NPs delivered systemically exhibit reduced recognition and clearance by the reticuloendothelial system compared to other commonly used polymeric NPs. Results demonstrated that PLA-HPG NPs retain their ideal qualities in the CSF space as well, with the hyperbranched structure forming a steric barrier around the NPs that extends circulation time and allows for greater tumor site accumulation. PLA-HPG NPs showed less than 15% accumulation in clearance organs at all time points as measured via PET/CT. The highest non-CNS accumulation occurred in the cervical lymph nodes, instead of the liver or spleen. Surprisingly, greater transfer from CSF to systemic clearance in tumor bearing mice compared to tumor-free mice was observed, which may be due to an abnormally leaky vasculature and dysfunctional lymphatic draining within the tumor microenvironment. Significant accumulation of NPs in the meninges of tumor-bearing mice, as well as at the site of tumor in the cerebellum, was also observed. It is unclear whether the NPs are engulfed by tumor-associated immune cells in the brain before trafficking to the meninges, or whether NPs arrive at the meninges and are then taken up by resident immune-cell types. It is likely a combination of both pathways, bulk CSF flow draining to the meninges and then to the cervical LNs, and active trafficking of NP-associated immune cells that represent an important drainage route for NPs in the CSF to the LNs.

With controlled release of drug, long-term retention of NPs can lead to prolonged drug exposure at site of tumor, and a means of improving the overall half-life of drug following intra-CSF administration. In addition, because activity in the CNS is at least 75% of total activity at all time points measured, there is a greatly reduced risk of widespread systemic toxicity. PARP inhibitors are limited by BBB penetration and widespread toxicity. PARPi were initially developed to sensitize tumor cells to conventional DNA damaging agents, and increasing evidence shows that PARPi is effective at sensitizing cells to radiotherapy, to temozolomide, and to topoisomerase poisons and inhibitors. However, efforts in clinic to use PARPi in combination have been marred by the high toxicity profile, and no PARPi has been approved for combination use. For pediatric CNS tumors, veliparib is the most clinically advanced PARPi due to its ability to cross the BBB. It has been evaluated in combination with temozolomide and with temozolomide plus radiotherapy, but difficulty in dose-escalating without causing toxicity and the absence of a survival benefit have slowed progress. TMZ sensitization is induced by PARP1 trapping, which is consistent with the failure of veliparib, which has relatively poor PARP1 trapping ability. In comparison, Talazoparib (BMN-673) is a potent PARP1 trap, but is constrained by its inability to bypass the BBB in meaningful quantities. Studies with BMN-673 are the first known preclinical study of delivering PARPi intrathecally, and unacceptable levels of toxicity in the free drug both alone and in combination with TMZ were found. Nanoencapsulation significantly improves the therapeutic index of BMN-673. Significantly higher doses (10×) with less systemic toxicity compared to free drug, as measured by blood cell counts, weight loss, and organ toxicity, were possible. Single-agent efficacy in an orthotopic model of MB was measured by giving equitoxic doses of either BMN-673 or BMN-NPs, and it was observed that only the encapsulated BMN NPs led to consistent tumor regression and an overall decrease in leptomeningeal spread. In addition, BMN-NPs were administered in conjunction with low-dose TMZ, and it was observed that this combination led to a durable response and was well-tolerated by the mice. This highlights the applicability of this approach and the potential to overcome the high tumor heterogeneity that is frequently observed in MB. The data indicates that delivery of NPs directly into the CSF enhances NP and hence drug exposure to cancer cells, resulting in favorable anti-tumor effects while minimizing damage in healthy tissue. In the clinic, repeat administration of NPs, such as through an Ommaya reservoir (intraventricular catheter used for delivery of drugs into the CSF), may improve therapeutic efficacy of therapy as compared to either i.v. administration or free drug administration. This integrated treatment approach could create new opportunities for PARPi combination therapies without compromising tolerability. In addition, this approach could lead to promising avenues of treatment for other diseases associated with extensive leptomeningeal spread, such as leptomeningeal metastases from primary malignancies such as lung, breast, and melanoma cancer.

1. A formulation comprising nanoparticles comprising a core of a hydrophobic polymer and a shell of hyperbranched polyglycerol having tissue adhesive functional groups thereon, the nanoparticles comprising therapeutic, prophylactic or diagnostic agents, in a dosage and pharmaceutically acceptable carrier for intrathecal delivery to the central nervous system.

2. The formulation of claim 1 comprising chemotherapeutic agents.

3. The formulation of claim 1 having a median diameter between 10 and 500 nm, more preferably between 25 and 250 nm, most preferably between 100 and 250 nm.

4. The formulation of claim 1 comprising a diagnostic agent.

5. The formulation of claim 1 wherein the adhesive functional groups are selected from the group consisting of aldehydes, amines, oximes, and O-substituted oximes.

6. The formulation of claim 1 wherein the functional group is an aldehyde.

7. The formulation of claim 2 wherein the chemotherapeutic agents are anti-cancer agents, preferably PARP inhibitors, preferably selected from the group consisting of olaparib, veliparib, CEP-8983 (II-methoxy-4,5,6,7-tetrahydro-IH-cyclopenta[a]pyrrolo[3,4-c]carbazole-I, 3 (2H)-dione) or a prodrug thereof (e.g. CEP-9722), rucaparib, E7016 (10-((4-hydroxypiperidin-I-yl)methyl) chromeno-[4,3,2-de]phthalazin-3 (2H)-one), INO-1001 (4-phenoxy-3-pyrrolidin-I-yl-5-sulfamoyl-benzoic acid), niraparib, talazoparib (BMN673), NU1025 (8-hydroxy-2-methylquinazolin-4 (3H)-one), 1,5-dihydroiso quinoline, 4-amino-1,8-naphthalimide, 2-nitro-6[5H]phenanthridinone, PD128763, and analogues, isosteres, and derivatives thereof.

8. The formulation of claim 1 wherein the hydrophobic polymer is a polyhydroxy acid, preferably poly(lactic acid) or poly(lactide-co-glycolide).

9. The formulation of claim 2 comprising a combination of a PARP inhibitor and temozolomide.

10. A method of delivering therapeutic, prophylactic or diagnostic agent to the central nervous system comprising administering the agent intrathecally in a formulation comprising nanoparticles comprising a core of a hydrophobic polymer, preferably a polyhydroxy acid, more preferably poly(lactic acid) or poly(lactide-co-glycolide), and a shell of hyperbranched polyglycerol having tissue adhesive functional groups thereon, in a dosage and pharmaceutically acceptable carrier for intrathecal administration.

11. The method of claim 10 wherein the agents are chemotherapeutic agents for treatment of cancers of the central nervous system, preferably leptomeningeal tumors like leptomeningeal metastasis and seeding tumors such as medulloblastoma.

12. The method of claim 10 wherein the nanoparticles comprise PARP inhibitors, preferably selected from the group consisting of olaparib, veliparib, CEP-8983 (II-methoxy-4,5,6,7-tetrahydro-IH-cyclopenta[a]pyrrolo[3,4-c]carbazole-I, 3 (2H)-dione) or a prodrug thereof (e.g. CEP-9722), rucaparib, E7016 (10-((4-hydroxypiperidin-1-yl)methyl) chromeno-[4,3,2-de]phthalazin-3 (2H)-one), INO-1001 (4-phenoxy-3-pyrrolidin-I-yl-5-sulfamoyl-benzoic acid), niraparib, talazoparib (BMN673), NU1025 (8-hydroxy-2-methylquinazolin-4 (3H)-one), 1,5-dihydroiso quinoline, 4-amino-1,8-naphthalimide, 2-nitro-6[5H] phenanthridinone, PD128763, and analogues, isosteres, and derivatives thereof.

13. The method of claim 10 comprising administering to a patient with a tumor a combination of a PARP inhibitor and temozolomide.

14. The method of claim 9 wherein the nanoparticles have a median diameter between 10 and 500 nm, more preferably between 25 and 250 nm, most preferably between 100 and 250 nm.

15. The method of claim 10 wherein the nanoparticles comprise a diagnostic agent.

16. The method of claim 10 wherein the nanoparticles comprise adhesive functional groups selected from the group consisting of aldehydes, amines, oximes, and O-substituted oximes, preferably aldehydes and/or amine functional groups.

17. The nanoparticles of claim 10 wherein the functional group is an aldehyde.

18. The method of claim 10 further comprising administering radiation to the brain after administration of the nanoparticles.
